SEQUENCE LISTING (29 sequences)

SEQ ID NO: 1 - DNA, 4308 bp, Artificial Sequence
  Cas9-P4
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctagcccggaagataaaattgctcagctgaaacaaaaaatccaagcgctgaaacaggaaaaccagcagctggaagaggaaaacgccgcactggaatatggttaa

SEQ ID NO: 2 - DNA, 4308 bp, Artificial Sequence
  Cas9-P3
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctcttccccggaagatgagatccagcaactggaagaagaaatcgctcagctggaacagaaaaacgcagcgctgaaagagaaaaaccaggcgctgaaatacggttaa

SEQ ID NO: 3 - DNA, 4293 bp, Artificial Sequence
  Cas9-N5
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctgagatcgccgccctggaggccaagatcgccgccctgaaggccaagaacgccgccctgaaggccgagatcgccgccctggaggcctaa

SEQ ID NO: 4 - DNA, 4293 bp, Artificial Sequence
  Cas9-N6
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctaagatcgccgccctgaaggccgagatcgccgccctggaggccgagaacgccgccctggaggccaagatcgccgccctgaaggcctaa

SEQ ID NO: 5 - DNA, 4305 bp, Artificial Sequence
  Cas9-P3S
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctagccccgaggacgagatccagcagctggaggaggagatcagccagctggagcagaagaacagccagctgaaggagaagaaccagcagctgaagtactaa

SEQ ID NO: 6 - DNA, 4305 bp, Artificial Sequence
  Cas9-P4S
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctagccccgaggacaagatcagccagctgaagcagaagatccagcagctgaagcaggagaaccagcagctggaggaggagaacagccagctggagtactaa

SEQ ID NO: 7 - DNA, 4305 bp, Artificial Sequence
  Cas9-AP4
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctagccccgaggacgagctggccgccaacgaggaggagctgcagcagaacgagcagaagctggcccagatcaagcagaagctgcaggccatcaagtactaa

SEQ ID NO: 8 - DNA, 957 bp, Artificial Sequence
  P4-EXOIII
agcccggaagataaaattgctcagctgaaacaaaaaatccaagcgctgaaacaggaaaaccagcagctggaagaggaaaacgccgcactggaatatggtggcggctctggcggcggctccggaggctctgatccaaaaaagaagagaaaggtaaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggacatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgctaa

SEQ ID NO: 9 - DNA, 957 bp, Artificial Sequence
  P3-EXOIII
tccccggaagatgagatccagcaactggaagaagaaatcgctcagctggaacagaaaaacgcagcgctgaaagagaaaaaccaggcgctgaaatacggtggcggctctggcggcggctccggaggctctgatccaaaaaagaagagaaaggtaaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggacatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgctaa

SEQ ID NO: 10 - DNA, 939 bp, Artificial Sequence
  N5-EXOIII
gagatcgccgccctggaggccaagatcgccgccctgaaggccaagaacgccgccctgaaggccgagatcgccgccctggaggccggcggctctggcggcggctccggaggctctgatccaaaaaagaagagaaaggtaaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggacatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 11 - DNA, 939 bp, Artificial Sequence
  N6-EXOIII
aagatcgccgccctgaaggccgagatcgccgccctggaggccgagaacgccgccctggaggccaagatcgccgccctgaaggccggcggctctggcggcggctccggaggctctgatccaaaaaagaagagaaaggtaaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggacatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 12 - DNA, 951 bp, Artificial Sequence
  P3S-EXOIII
agccccgaggacgagatccagcagctggaggaggagatcagccagctggagcagaagaacagccagctgaaggagaagaaccagcagctgaagtacggcggctctggcggcggctccggaggctctgatccaaaaaagaagagaaaggtaaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggacatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 13 - DNA, 951 bp, Artificial Sequence
  P4S-EXOIII
agccccgaggacaagatcagccagctgaagcagaagatccagcagctgaagcaggagaaccagcagctggaggaggagaacagccagctggagtacggcggctctggcggcggctccggaggctctgatccaaaaaagaagagaaaggtaaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggacatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 14 - DNA, 951 bp, Artificial Sequence
  AP4-EXOIII
agccccgaggacgagctggccgccaacgaggaggagctgcagcagaacgagcagaagctggcccagatcaagcagaagctgcaggccatcaagtacggcggctctggcggcggctccggaggctctgatccaaaaaagaagagaaaggtaaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggacatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 15 - DNA, 840 bp, Artificial Sequence
  P3-TREX2
tccccggaagatgagatccagcaactggaagaagaaatcgctcagctggaacagaaaaacgcagcgctgaaagagaaaaaccaggcgctgaaatacggtggcggctctggcggcggctccggaggctctatgtctgagccacctcgggctgagacctttgtattcctggacctagaagccactgggctcccaaacatggaccctgagattgcagagatatccctttttgctgttcaccgctcttccctggagaacccagaacgggatgattctggttccttggtgctgccccgtgttctggacaagctcacactgtgcatgtgcccggagcgcccctttactgccaaggccagtgagattactggtttgagcagcgaaagcctgatgcactgcgggaaggctggtttcaatggcgctgtggtaaggacactgcagggcttcctaagccgccaggagggccccatctgccttgtggcccacaatggcttcgattatgacttcccactgctgtgcacggagctacaacgtctgggtgcccatctgccccaagacactgtctgcctggacacactgcctgcattgcggggcctggaccgtgctcacagccacggcaccagggctcaaggccgcaaaagctacagcctggccagtctcttccaccgctacttccaggctgaacccagtgctgcccattcagcagaaggtgatgtgcacaccctgcttctgatcttcctgcatcgtgctcctgagctgctcgcctgggcagatgagcaggcccgcagctgggctcatattgagcccatgtacgtgccacctgatggtccaagcctcgaagcctga

SEQ ID NO: 16 - DNA, 837 bp, Artificial Sequence
  P4-TREX2
agcccggaagataaaattgctcagctgaaacaaaaaatccaagcgctgaaacaggaaaaccagcagctggaagaggaaaacgccgcactggaatatggtggcggctctggcggcggctccggaggctcttctgagccacctcgggctgagacctttgtattcctggacctagaagccactgggctcccaaacatggaccctgagattgcagagatatccctttttgctgttcaccgctcttccctggagaacccagaacgggatgattctggttccttggtgctgccccgtgttctggacaagctcacactgtgcatgtgcccggagcgcccctttactgccaaggccagtgagattactggtttgagcagcgaaagcctgatgcactgcgggaaggctggtttcaatggcgctgtggtaaggacactgcagggcttcctaagccgccaggagggccccatctgccttgtggcccacaatggcttcgattatgacttcccactgctgtgcacggagctacaacgtctgggtgcccatctgccccaagacactgtctgcctggacacactgcctgcattgcggggcctggaccgtgctcacagccacggcaccagggctcaaggccgcaaaagctacagcctggccagtctcttccaccgctacttccaggctgaacccagtgctgcccattcagcagaaggtgatgtgcacaccctgcttctgatcttcctgcatcgtgctcctgagctgctcgcctgggcagatgagcaggcccgcagctgggctcatattgagcccatgtacgtgccacctgatggtccaagcctcgaagcctga

SEQ ID NO: 17 - DNA, 2668 bp, Artificial Sequence
  P3-EXO1
tccccggaagatgagatccagcaactggaagaagaaatcgctcagctggaacagaaaaacgcagcgctgaaagagaaaaaccaggcgctgaaatacggtggcggctctggcggcggctccggaggctctgcgatcgccatggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaaggggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctatgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaa

SEQ ID NO: 18 - DNA, 2668 bp, Artificial Sequence
  P4-EXO1
agcccggaagataaaattgctcagctgaaacaaaaaatccaagcgctgaaacaggaaaaccagcagctggaagaggaaaacgccgcactggaatatggtggcggctctggcggcggctccggaggctctgcgatcgccatggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaaggggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctatgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaa

SEQ ID NO: 19 - DNA, 20 bp, artificial sequence
  targeting sequence in eGFP genomic region
ggcgagggcgatgccaccta

SEQ ID NO: 20 - DNA, 20 bp, artificial sequence
  human EMX1 gene targeting sequence
gagtccgagcagaagaagaa

SEQ ID NO: 21 - DNA, 20 bp, artificial sequence
  human VEGF gene targeting sequence
ggtgagtgagtgtgtgcgtg

SEQ ID NO: 22 - DNA, 20 bp, artificial sequence
  human MYD88 gene targeting sequence
ggctgagaagcctttacagg

SEQ ID NO: 23 - DNA, 20 bp, artificial sequence
  human ANKRD52 gene targeting sequence
actgtaaaggctgctctccc

SEQ ID NO: 24 - DNA, 20 bp, artificial sequence
  human FUT9 gene target sequence
tgcagaggagcctttacatg

SEQ ID NO: 25 - DNA, 20 bp, artificial sequence
  human PSKH2 gene targeting sequence
gccagacaaggctttacagg

SEQ ID NO: 26 - DNA, 20 bp, artificial sequence
  human BCR-ABL fusion gene targeting sequence
gacctgtcttttagacaggc

SEQ ID NO: 27 - DNA, 40 bp, artificial sequence
  equivalent Cas9 protein expression - pcDNA3
ggaggctgagaagcctttacaggtggccgctgtagacagc

SEQ ID NO: 28 - DNA, 31 bp, artificial sequence
  equivalent Cas9 protein expression - Cas9
ggaggctgagaaggtggccgctgtagacagc

SEQ ID NO: 29 - DNA, 26 bp, artificial sequence
  equivalent Cas9 protein expression - Cas9+EXO3
ggaggcaggtggccgctgtagacagc

(19) United States
(12) Patent Application Publication
(10) Pub. No.: US 2022/0307011 A1
(43) Pub. Date: Sep. 29, 2022

(54) COILED-COIL MEDIATED TETHERING OF CRISPR/CAS AND EXONUCLEASES FOR ENHANCED GENOME EDITING

(71) Applicant: Kemijski Institut, Ljubljana (SI)

(72) Inventors: Roman JERALA, Ljubljana (SI); Dusko LAINSCEK, Ljubljana (SI)

(73) Assignee: Kemijski Institute, Ljubljana (SI)

(21) Appl. No.: 17/636,450

(22) PCT Filed: Aug. 19, 2020
(86) PCT No.: PCT/EP2020/073143

§ 371 Date: Feb. 18, 2022

(30) Foreign Application Priority Data
Aug. 20, 2019 (EP) 19192490.1

(51) Int. Cl. C12N 15/10

(52) U.S. Cl. CPC C12N 15/1055; C12N 15/102; C12Y 305/04005; C07K 2319/80; C12N 2310/20

(57) ABSTRACT

The invention provides a method for enhanced genome engineering using CRISPR/Cas system tethered to exonucleases via different systems. By connecting Cas9 and exonucleases via heterodimeric peptides that form coiled-coils or via heterodimerization systems, greater indel mutations at desired genomic region occurs, resulting in higher genome editing rate. This novel improved CRISPR/Cas system can be exploited in different cell origins and organisms in various fields, where genome engineering is required and desired.

### FIELD OF THE INVENTION

Heterodimerization of Cas9 protein, part of CRISPR/Cas system, with exonucleases via peptides that form coiled-coils increases the rate of occurrence of indel mutations in genome region of interests in cells resulting in higher degree of genome engineering that is exploited in all areas where genome editing is required.

### BACKGROUND OF THE INVENTION

The CRISPR (Clustered Regularly Interspaced Short Palindromic Repeats)/Cas (CRISPR-associated) system that is used as a tool for genome editing and derives from bacteria and archaea as a part of their adaptive immune system to fight infectious viruses. When prokaryotes are invaded with viruses, short viral DNA segments are integrated into CRISPR locus, which is the key for CRISPR immunity. Upon second accounter with viruses, RNA that includes viral sequence, from CRISPR locus is transcribed. That RNA is complimentary to viral DNA and mediates targeting of Cas9 protein to the sequence in genome. This specific targeting results in DNA cleavage and thereby silencing the viral target,

This specific targeting and DNA cleavage at desired genome region made CRISPR/Cas system as a potent novel tool for genome editing. Cas9 protein or its variants are guided by gRNA (guide RNA) to its complimentary specific site or region of interest (ROI) within the genome. The only necessity for Cas9 recognition is short PAM (protospacer adjacent motif) sequence presence within the genome. Depending on the Cas9 origin, different PAM sequences are recognized. After specific Cas9 targeting to ROI site-specific double strand breaks (DSBs) occur, which vary in length and in frequency. Repair of DSBs and their manipulation lead to knockout or knockin of gene of interests (GOI).

DSBs are then repaired with cell repair mechanisms, mostly with error-prone classical-Non-homologous end joining (c-NHEJ), which is DNA resection independent. Other repair mechanisms, like alt-EJ (alternative end joining), HR (homologous recombination), SSA (single-strand annealing) work in DNA resection dependent manner. C-NHEJ mainly introduces few nucleotides long insertion-deletion (indel) mutations that can lead to functional gene inactivation by either frameshift or deletions resulting in certain percentage or degree of knockout of GOI. C-NHEJ repairs Cas9 mediated blunt-end DSB via simple blunt end sequence-homology independent ligation process. Alternatively, DSBs can be resected, leaving 3′ or 5′ single-stranded DNA (ssDNA) overhangs. When DSBs via HR are repaired a certain DNA template as a source for repair is needed. Usually sister chromatid get strand invaded in typically error-free process, resulting in site-specific integration. This is often exploited for knockin production where included exogenous DNA reacts as a template. ssDNA overhangs can be also repaired via mutagenic repair mechanism, namely SSA, where annealing at large homologies occurs, resulting in larger deletions with no insertions. Alt-EJ uses annealing at microhomologies, resulting in mutagenic indel mutations.

Depending on the size and frequency of indel mutations, higher rates of knockout can be achieved. Greater genome rearrangements occurs when additional DNA resection after DSBs are carried out. DNA resection and subsequent DNA repair is used for elevated genome editing using CRISPR/Cas regarding the efficiency. Coexpression of certain exonucleases (e.g. human EXO1, TREX2) with CRISPR/Cas system can increase the size of mutations resulting in higher frequency of knockin or knockout of GOI.

### SUMMARY OF THE INVENTION

The present invention provides an implementation for CRISPR/Cas system for greater efficiency regarding genome modification and editing based on tethering of Cas9 protein with exonucleases via different heterodimerization systems. Tethering of CRISPR/Cas system and exonucleases result in higher percentage or degree of genome modification for GOI or recisse DNA to the extent to allow higher homologies for greater percentage of knockin.

This disclosure provides new system of improved genome editing by the use of CRISPR/Cas system. Genome editing herein encompasses DNA cleavage and sequential repair. DSBs are formed by protein Cas9 or its variants or Cas9 nickase (HNH-mutant nickase or RuvC-mutant nickase) that are guided via one or two gRNA to specific genome sites. This system for enhanced genome editing can be used in living mammalian and non-mammalian (e.g. plant) cells.

Heterodimerization of CRISPR/Cas system and certain exonucleases is achieved by the use of heterodimerizing peptides that form coiled-coils (CC) that are expressed with Cas9 protein at its C- or N- terminus and with certain exonucleases at its C- or N-terminus thus Cas9 gets tethered with certain exonucleases. Tethering of Cas9 with certain exonucleases brings exonucleases into close proximity of DSB to influence DNA recession. Collocation of certain exonuclease at DSB improves DNA recession thus making longer 3′ and/or 5′ DNA overhangs. Longer DNA overhangs generates greater non-homologies between upper and lower DNA strands thus making larger indel mutations after DSB repairs at higher degree. Heterodimerization of Cas9 protein or its variants or Cas9 nickase and certain exonucleases is achieved due to the dimer forming peptides. Heterodimerizing peptides express coiled coil structural motif, a combination of hydrophobic and electrostatic interactions between amino acid residues within heptads of alpha-helices.

This disclosure presents tethering of CRISPR/Cas system and certain exonucleases via additional heterodimerization systems. System is based on the selection of heterodimerization (HD) domains (different protein-protein partners; HD system), which allows regulated expression in cells upon presence of one or more inductors that can provide external regulator signals. Inductors for heterodimerization can be light (e.g. Blue light, red light etc.) or chemical nature depending on the use of protein-protein partner pair. Such chemical inductors can be small molecules, such as rapalog, gibberellin and/or abscisic acid.

Protein partners of the HD system dimerize into a functional form when the appropriate heterodimerizing signal is present. One partner of the HD system is connected to a Cas9 protein or its variants or Cas9 nickase. The other partner of the selected heterodimerization system is coupled with certain exonuclease. By the addition of an external light at the appropriate wavelength or small chemical inductor or regulator, respectively, (for example a rapalog, gibberellin or abscisic acid) that acts to promote heterodimerization, two partners of an HD system dimerize into a heterodimer. Heterodimerization of two protein partners of HD systems allows close proximity of certain exonuclease at the point of DSB, which is CRISPR/Cas system mediated. Juxtaposition of certain exonuclease at DSB enhances DNA recession thus making longer 3′ and/or 5′ DNA overhangs. Longer DNA overhangs generates greater non-homologies between upper and lower DNA strands thus making larger indel mutations after DSB repairs at higher degree.

The invention provides modified CRSIPR/Cas system based on the use of HD with certain exonucleases. Enhanced genome editing includes knockout and knockin for desired GOI at determined gene locus in mammalian and non-mammalian cells.

### FIGURE LEGENDS

FIG. 1: A schematic diagram of action of the method for enhanced genome engineering using CRISPR/Cas system according to the invention, tethered to exonucleases via heterodimeric peptides that form coiled-coils. A) By using simple CRISPR/Cas system that consists out of Cas9 protein and gRNA DSB are formed, which are then repaired via cell repair mechanisms, resulting in certain degree or percentage of genome editing. B) By using cooexpresion of simple CRISPR/Cas system and certain exonuclease (e.g. EXOIII) DSBs are formed. The exposed DNA strands are then additionally recised via certain exonucleases, creating DNA overhangs in 5′ or 3′ manner. Again, DSBs with established DNA overhangs are repaired via cell repair mechanisms, resulting in higher degree or percentage of genome editing compared to simple CRISPR/Cas system. C) By using CRISPR/Cas system tethered to certain exonuclease (e.g. EXOIII) via heterodimeric peptides that form coiled-coils DSBs are formed. The exposed DNA strands are then additionally recised via certain exonucleases, creating even larger DNA overhangs compared to overhangs, created in system, where CRISPR/Cas and exonucleases are coexpressed. Again, DSBs with established DNA overhangs are repaired via cell repair mechanisms, resulting in even higher degree or percentage of genome editing compared to simple CRISPR/Cas system or system, where CRISPR/Cas and exonucleases are coexpressed.

FIG. 2: Genome editing in eGFP genome region. By coexpression of Cas9 and gRNA, directed towards eGFP genomic region eGFP genome region is modified. Greater genome editing occurred when Cas9, gRNA were coeexpressed with exonucleases, wherein EXO1, EXOIII, FEN1, WRN or mTREX2 is at least one of the exonuclease used. eGFP genome editing resulted in eGFP fluorescence decrease.

FIG. 3: Genome editing in human MYD88 genome region. By coexpression of Cas9 and gRNA, directed towards human MYD88 genomic region human MYD88 genome region is modified. Greater genome editing occurred when Cas9, gRNA were coeexpressed with exonucleases, wherein EXO1, EXOIII, or mTREX2 is at least one of the exonuclease used. Bigger genome editing occurred when CRISPR/Cas system tethered to certain exonucleases via heterodimeric coiled-coil forming peptides were used, wherein Cas9-P3 or Cas9-P4 are one of the Cas9 variants and P3-EXO1, P4-EXO1, P3-EXOIII, P4-EXOIII, P3-mTREX2 or P4-mTREX2 are one of the variants of expressed exonucleases. A) Human MYD88 genome editing resulted in indel mutations, detected via T7E1 assay. B) Indel detection (%) of MYD88 genome region engineering.

FIG. 4: Genome editing in human VEGF genome region. By coexpression of Cas9 and gRNA, directed towards human VEGF genomic region human VEGF genome region is modified. Greater genome editing occurred when Cas9, gRNA were coeexpressed with exonucleases, wherein EXO1, EXOIII, or mTREX2 is at least one of the exonuclease used. Bigger genome editing occurred when CRISPR/Cas system tethered to certain exonucleases via heterodimeric coiled-coil forming peptides were used, wherein Cas9-P3 or Cas9-P4 are one of the Cas9 variants and P3-EXO1, P4-EXO1, P3-EXOIII, P4-EXOIII, P3-mTREX2 or P4-mTREX2 are one of the variants of expressed exonucleases. A) Human VEGF genome editing resulted in indel mutations, detected via T7E1 assay. B) Indel detection (%) of VEGF genome region engineering.

FIG. 5: Genome editing in human EMX1 genome region. By coexpression of Cas9 and gRNA, directed towards human EMX1 genomic region human EMX1 genome region is modified. Greater genome editing occurred when Cas9, gRNA were coeexpressed with exonucleases, wherein EXO1, EXOIII, or mTREX2 is at least one of the exonuclease used. Bigger genome editing occurred when CRISPR/Cas system tethered to certain exonucleases via heterodimeric coiled-coil forming peptides were used, wherein Cas9-P3 or Cas9-P4 are one of the Cas9 variants and P3-EXO1, P4-EXO1, P3-EXOIII, P4-EXOIII, P3-mTREX2 or P4-mTREX2 are one of the variants of expressed exonucleases. A) Human EMX1 genome editing resulted in indel mutations, detected via T7E1 assay. B) Indel detection (%) of EMX1 genome region engineering.

FIG. 6: Equivalent Cas9 protein expression. Cas9 protein is expressed in the same manner when only Cas9 is expressed or cooexpressed with exonucleases, wherein EXO1, EXOIII, or mTREX2 is at least one of the exonuclease used. The same Cas9 expression is observed when CRISPR/Cas system tethered to certain exonucleases via heterodimeric coiled-coil forming peptides were used, wherein Cas9-P3 or Cas9-P4 are one of the Cas9 variants and P3-EXO1, P4-EXO1, P3-EXOIII, P4-EXOIII, P3-mTREX2 or P4-mTREX2 are one of the variants of expressed exonucleases. Cas9 expression is detected via Western blot and immunodetection.

FIG. 7: Genome editing in human MYD88 genome region. By coexpression of Cas9 and gRNA, directed towards human MYD88 genomic region human MYD88 genome region is modified. Greater genome editing occurred when Cas9, gRNA were coeexpressed with exonucleases, wherein EXO1, EXOIII, or mTREX2 is at least one of the exonuclease used. Bigger genome editing occurred when CRISPR/Cas system tethered to certain exonucleases via heterodimeric coiled-coil forming peptides were used, wherein Cas9-P3 or Cas9-P4 are one of the Cas9 variants and P3-EXO1, P4-EXO1, P3-EXOIII, P4-EXOIII, P3-mTREX2 or P4-mTREX2 are one of the variants of expressed exonucleases. Next generation sequencing was performed to detect the size of edited genome region.

FIG. 8: Absence of human MYD88 “off target” genome editing due to increased CRISPR/Cas action. By coexpression of Cas9 and gRNA, directed towards human MYD88 genomic region human MYD88 genome region is modified. Greater genome editing occurred when Cas9, gRNA were coeexpressed with exonucleases, wherein EXO1, EXOIII, or mTREX2 is at least one of the exonuclease used. Bigger genome editing occurred when CRISPR/Cas system tethered to certain exonucleases via heterodimeric coiled-coil forming peptides were used, wherein Cas9-P3 or Cas9-P4 are one of the Cas9 variants and P3-EXO1, P4-EXO1, P3-EXOIII, P4-EXOIII, P3-mTREX2 or P4-mTREX2 are one of the variants of expressed exonucleases. Three predicted coding regions of human MYD88 gene targeting gRNA “off-target” sites were screened via T7E1 assay. A) Predicted “off-target” site 1 (human ANKRD52 gene) genome editing did not result in indel mutations, detected via T7E1 assay. B) Predicted “off-target” site 2 (human FUT9 gene) genome editing did not result in indel mutations, detected via T7E1 assay. C) Predicted “off-target” site 3 (human PSKH2 gene) genome editing did not result in indel mutations, detected via T7E1 assay.

FIG. 9: Reduction in bioluminescence signal (photons/second) of K562-fLUC cells. gRNA targeting BCR-ABL chromosomal translocation was designed. CRISPR/Cas genome editing at BCR-ABL chromosomal translocation resulted in cell death, detected via drop of bioluminescence signal. By coexpression of Cas9 and gRNA, directed towards human BCR-ABL chromosomal translocation genomic region human BCR-ABL genome region is modified. Greater genome editing occurred when Cas9, gRNA were coeexpressed with exonucleases, wherein EXO1, EXOIII, or mTREX2 is at least one of the exonuclease used. Bigger genome editing occurred when CRISPR/Cas system tethered to certain exonucleases via heterodimeric coiled-coil forming peptides were used, wherein Cas9-P3 or Cas9-P4 are one of the Cas9 variants and P3-EXO1, P4-EXO1, P3-EXOIII, P4-EXOIII, P3-mTREX2 or P4-mTREX2 are one of the variants of expressed exonucleases.

FIG. 10: Genome editing in human BCR-ABL genome region. By coexpression of Cas9 and gRNA, directed towards human BCR-ABL genomic region human BCR-ABL8 genome region is modified. Greater genome editing occurred when Cas9, gRNA were coeexpressed with exonucleases, wherein EXOIII is at least one of the exonuclease used. Bigger genome editing occurred when CRISPR/Cas system tethered to certain exonucleases via heterodimeric coiled-coil forming peptides were used, wherein Cas9-P3 are one of the Cas9 variants and P4-EXOIII are one of the variants of expressed exonucleases. Even greater genome editing occurred when Cas9-N5 and N6-EXOIII variants were used, showing stronger heterodimerization affinity of peptides that form coiled-coils.

### DEFINITIONS

The term CRISPR/Cas system, as used herein relates to one or more gRNA that guides catalytically active Cas9 or its variants or inactive nickase Cas9 or its variants protein to the desired genomic site where DSB formations are formed through specific Cas9 action after appropriate PAM recognition. Cas9 protein in this invention is fused with heterodimerization system at N- or C-terminus that allows tethering with certain exonucleases.

Cas protein, as used herein relates to Cas9 and its variants and homologous derived from Streptococcus pyogenes that recognizes NGG or NAG PAM. This invention relies also on all other DNA catalytically active or inactive endonucleases (dCas9) or nickases derived from all types of CRISPR system.

APOBEC polypeptide, as used herein relates to proteins that have deaminase activity and are connected with Cas9 and its variants and causes single base editing.

Genome editing, as used herein relates to any modification to genomic and/or non-genomic DNA due to CRISPR/Cas system and its efficiency to cause DSBs. Sequentially single base editing, knockin or knockout cells and/or organisms are processed.

Organism, as used herein relates to any living organism.

The term exonucleases as used herein, relates to any sort of enzymes that are able to cleave nucleotides from polynucleotide chain in 3′ and/or 5′ direction and are connected to heterodimerization system at N- or C-terminus.

The term “heterodimerization system”, as used herein, refers to a pair of peptides or peptide domains that form coiled-coils or that connect in the presence of an inductor or a signal for dimerization. The term “heterodimerization”, as used herein, refers to protein domains or peptide pairs that connect to other domains of a different type through covalent or non-covalent interactions. The term “constitutive dimerization domains”, as used herein, refers to dimerization domains that connect themselves independently, such as, for example, coiled coils. The term “inducible heterodimerization domain”, as used herein, refers to heterodimerization domains that merge only in the presence of a heterodimerization inductor.

The term “heterodimerization inductor” refers to a signal, a chemical or non-chemical ligand, which cause the heterodimerization of protein domains that do not have intrinsic affinity with each other and cannot be connected independently in the absence of a heterodimedimerization ligand or signal. A heterodimerization inductor according to the invention can be a small molecule, such as rapalog, abscisic acid, or gibberellin. According to this invention, a heterodimerization inductor can also be light with certain wavelength.

Light induced heterodimerization system, as used herein, refers to protein partners CIB1 or its homologous and CRY2PHR, derived from Arabidopsis thaliana or are produced synthetically and heterodimerize in the presence of blue light with certain wavelength, approximately 450 nm. Another blue light induced heterodimerization system is LOVpep and ePDZB protein partners, derived from Avena sativa. This invention also relates to 650 nm wavelength red light induced heterodimerization system, where protein partners PIF and PHYB or its synthetic homologous are used. Another light induced heterodimerization system that this invention relies is heterodimerization of FKF1 and GI protein partners from Gigantea sp. The term “chemical ligand”, as used herein, refers to a small molecule, such as rapamycin and its rapalogs that can bind to proteins, preferably to FKBP-FRB heterodimerization domains; to plant hormones, such as abscisic acid and giberellin that can bind to proteins, preferably to ABI-PYL1and GID-GAI1 heterodimerization domains.

The term peptide pair that form coiled coils, used herein refers to any peptide pair, heterodimerizing or homodimerizing peptides that express coiled coil structural motif, a combination of hydrophobic and electrostatic interactions between amino acid residues within heptads of alpha-helices that results in heterodimerization or homodimerization.

The term “orthogonal”, as used herein, refers to the characteristic of the components of the engineered CRISPR/Cas system tethered to certain exonucleases via different heterodimerization systems to take place separately, i.e., that the individual parts of different heterodimerization systems do not interact with parts of other heterodimerization systems of endogenous or exogenous signaling pathways. The main feature of the orthogonal system is that the input signal of each system leads to the heterodimerization independently of the presence or absence of other systems or their input and output signals.

The term “cell”, as used herein, refers to an eukaryotic or prokaryotic cell, a cellular or multicellular organism (cell line) cultured as a single cell entity that has been used as a recipient of nucleic acids and includes the daughter cells of the original cell that has been genetically modified by the inclusion of nucleic acids. The term refers primarily to cells of higher developed eukaryotic organisms, preferably vertebrates, preferably mammals. This invention relies also on non-vertebrates cells, preferably plant cells.

The term “cells” also refers to human cell lines and plant cells. Naturally, the descendants of one cell are not necessarily completely identical to the parents in morphological form and its DNA complement, due to the consequences of natural, random or planned mutations. A “genetically modified host cell” (also “recombinant host cell”) is a host cell into which the nucleic acid has been introduced. The eukaryotic genetically modified host cell is formed in such a way that a suitable nucleic acid or recombinant nucleic acid is introduced into the appropriate eukaryotic host cell. The invention hereafter includes host cells and organisms that contain a nucleic acid according to the invention (transient or stable) bearing the operon record according to the invention. Suitable host cells are known in the field and include eukaryotic cells. It is known that proteins can be expressed in cells of the following organisms: human, rodent, cattle, pork, poultry, rabbits and the like. Host cells may include cultured cell lines of primary or immortalized cell lines.

The term “nucleic acids”, as used herein, refers to a polymeric form of nucleotides (ribonucleotides or deoxyribonucleotides) of any length and is not limited to single, double or higher chains of DNA or RNA, genomic DNA, cDNA, DNA-RNA hybrids, or polymers with a phosphorothioate polymer backbone made from purine and pyrimidine bases or other natural, chemical or biochemically modified, synthetic or derived nucleotide bases.

The term “protein”, as used herein, refers to the polymeric form of amino acids of any length, which expresses any function, for instance localizing to a specific location, localizing to specific DNA sequence, facilitating and triggering chemical reactions, transcription regulation.

The term “recombinant”, as used herein, means that a particular nucleic acid (DNA or RNA) is a product of various combinations of cloning, restriction and/or ligation leading to a construct having structurally coding or non-coding sequences different from endogenous nucleic acids in a natural host system.

The insertion of the vectors into the host cells is carried out by conventional methods known from the field of science, and the methods relate to transformation or transfection and include: chemically induced insertion, electroporation, micro-injection, DNA lipofection, cellular sonication, gene bombardment, viral DNA input, as well as other methods. The entry of DNA may be of transient or stable. Transient refers to the insertion of a DNA with a vector that does not incorporate the DNA of the invention into the cell genome. A stable insertion is achieved by incorporating DNA of the invention into the host genome. The insertion of the DNA of the invention, in particular for the preparation of a host organism having stably incorporated a nucleic acid, e.g. a DNA, of the invention, can be screened by the presence of markers. The DNA sequence for markers refers to resistance to antibiotics or chemicals and may be included on a DNA vector of the invention or on a separate vector.

The insertion of the CRISPR/Cas tethered with certain exonucleases via different heterodimerization systems are delivered to the cell as plasmid DNA or as mRNA or a proteins or as RNA:protein complexes where different parts of CRISPR/Cas system tethered with certain exonucleases via different heterodimerization systems can be as DNA or RNA or protein.

### DETAILED DESCRIPTION OF THE INVENTION

The invention relates to an enhanced CRISPR/Cas system that is made of a combination of two orthogonal protein partners of different heterodimerization (HD) systems or coiled-coil forming peptide pairs, connected with a Cas protein and certain exonucleases that reconstitute upon addition of an inductor of heterodimerization or via intrinsic affinity of peptide pairs that form coiled-coils. Functionally, enhanced CRISPR/Cas system influences the higher rate of genome editing based on larger insertion and/or deletion mutations that arise in higher percentage of genetically modified cells or organisms.

Chemical and non-chemical control over the activation of enhanced CRISPR/Cas system tethered to certain exonucleases via different heterodimerization systems of gene modified cells or organisms of this invention presents a novel way of gene editing in different research area, e.g. cancer therapy, new model organism production, new drug development etc.

The invention provides a combination comprising a first component (a) and a second component (b), wherein

a) the first component comprises an endonuclease capable of inducing a double stranded DNA break or a nickase capable of inducing a DNA nick, preferably a Cas protein or mutant thereof, fused with a first protein or protein domain, and

b) the second component comprises an exonuclease, fused to a second protein or protein domain,

wherein the first and the second components are capable of heterodimerizing with each other via interaction of the first protein or protein domain and the second protein or protein domain.

The combination of the invention represents a modified CRISPR/Cas system. The endonuclease of the first component (a) can in particular be a Cas protein, a Cas nickase or dCas9 protein. The second component (b) can in particular be an exonuclease or cytidine deaminase, in particular APOBEC protein.

According to a particular aspect of the invention, heterodimerization of the first protein or protein domain and the second protein or domain is inducible via a chemical or non-chemical signal. The (at least one) first protein or protein domain of the first component can be a single protein partner of a selected chemically or non-chemically inducible heterodimerization system (e.g. ABI-PYL1, FKBP-FRB or DmrC-DmrA, GAI-GID1) or non-chemically (e.g. CIBN-CRY2PHR, LOvpep-ePDZb, PIF-PHYB, FKF1-GI) that is genetically fused to a Cas protein or its variants; for example: CIBN or LOVpep or PIF or FKF1 or ABI or FKBP or GAI can be connected to a Cas protein or its variants (e.g. Cas9, Cas9nickase, Cpf1).

The second protein or protein domain of the second component can be a second protein partner of a selected heterodimerization system (e.g. ABI-PYL1, FKBP-FRB or DmrC-DmrA, GAI-GID1, CIBN-CRY2PHR, LOvpep-ePDZb, PIF-PHYB, FKF1-GI) that is genetically fused to a certain exonuclease (e.g. human EXO1, EXOIII E.coli, TREX2 etc.).

Addition of a chemical inductor of heterodimerization (HD) can trigger heterodimerization of the first component and the second component by heterodimerization of two protein partners (i.e. the at least one protein or protein domain and the second protein or protein domain), thereby resulting in a functional modified CRISPR/Cas system tethered with certain exonucleases, which can influence the genome modification of any GOI.

A chemical inductor for triggering heterodimerization of the at least one first protein or protein domain of the a) first component and the second protein or protein domain of the b) second component can be, e.g. rapalog (e.g. rapamycin) for triggering heterodimerization of a FKBP-FRB HD system (the at least one first protein domain is FKBP, the second protein domain is FRB), abscisic acid for triggering heterodimerization of a ABI-PYL1 HD system (the at least one first protein domain is ABI, the second protein domain is PYL1), gibberellin for triggering heterodimerization of a GID-GA1 HD system (the at least one first protein domain is GID, the second protein domain is GA1) or any other relevant physiological chemical signal.

Addition of a non-chemical inductor of heterodimerization (HD) can trigger heterodimerization of the first component and the second component by heterodimerization of two protein partners (i.e. the at least one protein or protein domain and the second protein or protein domain), thereby resulting in a functional modified CRISPR/Cas system tethered with certain exonucleases, which can influence the genome modification of any GOI.

A non-chemical inductor for triggering heterodimerization of the at least one first protein or protein domain of the a) first component and the second protein or protein domain of the b) second component can be light with appropriate wave length, e.g. blue light (e.g. 450 nm wave length) for triggering heterodimerization of a CIBN-CRY2PHR HD system (the at least one first protein domain is CIBN, the second protein domain is CRY2PHR) or LOVpep-ePDZb HD system (the at least one first protein domain is LOVpep, the second protein domain is ePDZb). Other inductor for light induced heterodimerization is red light (e.g. 650 nm wave length) for triggering heterodimerization of a PIF-PHYB HD system (the at least one first protein domain is PIF, the second protein domain is PHYB) or for triggering heterodimerization of a FKF1-GI HD system (the at least one first protein domain is FKF1, the second protein domain is GI) or any other relevant physiological non-chemical signal.

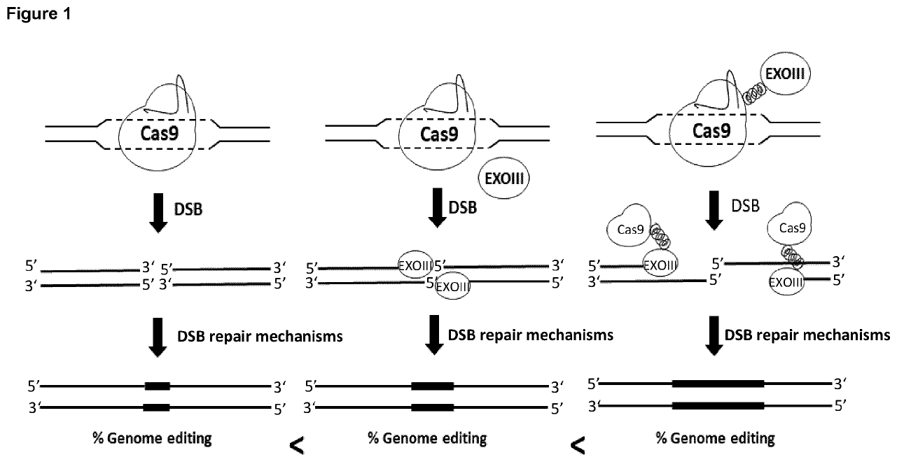

In a further embodiment of the invention, the heterodimerization between endonuclease or nickase (e.g. Cas protein or variant or mutant thereof) and certain exonuclease can be achieved using peptide pairs that form coiled-coils due to the intrinsic affinity and electrostatic interactions and thus bringing certain exonucleases into close proximity of DSB allowing exonuclease to additionally recise DNA in 3′ and/or 5′ manner. In this embodiment, enhancing the function of regular CRISPR/Cas system through additional DNA recizing with certain exonucleases can result in greater percentage of genome modification compared to normal CRISPR/cas system or system where CRISPR/Cas and certain exonucleases are cooexpressed in tested cell or organism (see, e.g. FIG. 1).

The first component of peptide pair coiled-coil induced heterodimerization system can be at least one peptide or peptide domain from peptide pair that form coiled-coils of a selected peptide pairs (e.g. P3-P4, N5-N6, P3S-P4S etc) that is genetically fused to a Cas protein or its variants; for example: P3, N5 or P3S can be connected to Cas9 protein or its variants (e.g. Cas9, Cas9nickase, Cpf1 etc.).

The second component of peptide pair coiled-coil induced heterodimerization system can be at least one peptide or peptide domain from peptide pair that form coiled-coils of a selected peptide pairs (e.g. P3-P4, N5-N6, P3S-P4S etc.) that is genetically fused to a certain exonucleases (human EXO1, EXOIII E.coli, TREX2 etc).

In a further embodiment of the invention, the selected pair of peptide pair that form coiled-coils are chosen based on their physical properties (e.g. Kd values, their orthogonality properties etc.). Peptide pairs that form coiled-coils can have strong heterodimerization properties: for example, peptide pair N5-N6 exhibit stronger heterodimerization affinity than peptide pair P3S-P4S. From this follows that percentage of genome editing in N5-N6 used heterodimerizing peptide pair that form coiled-coils for enhanced CRISPR/Cas system tethered with certain exonucleases is greater than for the example where P3S-P4S heterodimerizing peptide pair that form coiled-coils is used, that exhibit poor heterodimerization properties.

In a further embodiment, heterodimerization of peptide pairs, which form coiled-coils, can be obtained by using peptide pairs that heterodimerize in parallel (e.g. N5-N6, P3-P4 etc.) or anti-parallel (e.g.P3-AP4) manner.

In a further embodiment the use of heterodimerization systems for tethering CRISPR/Cas system with certain exonucleases, additional DNA recession with certain exonucleases and sequential improvement in genome editing does not enhance possible off-target activities of CRISPR/Cas system.

In a further embodiment, the modified CRISPR/Cas system tethered to certain exonucleases via different heterodimerization systems according to the invention comprises several repeats of the at least one first protein domain and/or the second protein domain, e.g. 10 repeats of a coiled coil. In this embodiment, the genome editing can be further enhanced. Several repeats (for example 10 repeats of coiled coil of a peptide pair) of the at least one first protein domain can then bind to several repeats (for example 10 repeats of coiled coil) of the second protein domain, which is fused to certain exonuclease. The first component comprising the at least one first protein domain having several repeats then heterodimerizes with the second component comprising the second protein domain having several repeats after the appropriate chemical inductor is added. In this embodiment, the repeats can act as constitutive dimerization domains that assemble upon the heterodimerization signal by the chemical inductor as orthogonal coiled coil pairs.

In a further embodiment, the at least one first protein domain of the chemical signal-inducible heterodimerization protein complex of the first component is selected from the group consisting of the pairs:
i) FKBP and FRB;
ii) FKBP and calcineurin catalytic subunit A (CnA);
iii) FKBP and cyclophilin;
iv) gyrase B (GyrB) and GyrB;
v) DmrA and DmrC;
vi) ABI and PYL1; and
vii) GAI and GID1, and
   the second protein domain of the second component is selected from the group consisting of the pairs:
i) FKBP and FRB;
ii) FKBP and calcineurin catalytic subunit A (CnA);
iii) FKBP and cyclophilin;
iv) gyrase B (GyrB) and GyrB;
v) DmrA and DmrC;
vi) ABI and PYL1; and
vii) GAI and GID1.

In a further embodiment, the at least one first protein domain of the light signal-inducible heterodimerization protein complex of the first component is selected from the group consisting of the pairs:
i) CIB1 and CRY2;
ii) LOV and PDZ;
iii) PIF and PHYB;
iv) FKF1-GI
v) UVR8-COP1,
   and the second protein domain of the second component is selected from the group consisting of the pairs:
i) CIB1 and CRY2;
ii) LOV and PDZ;
iii) PIF and PHYB;
iv) FKF1-GI
v) UVR8-COP1.

In a further embodiment of the invention, the at least one first protein domain and the second protein domain can be from two different proteins and can have orthologous properties.

The invention further provides an isolated nucleic acid molecule, or a combination of isolated nucleic acid molecules, comprising a first nucleic acid sequence encoding the first component (a) and a second nucleic acid sequence encoding the second component (b) of the combination according to the invention as defined above. The invention further provides a vector, preferably an expression vector, encoding the nucleic acid molecule(s) of the invention.

In particular, the invention provides isolated nucleic acid molecules comprising sequences comprising or consisting of SEQ ID NOs: 1-18, as per the enclosed sequence listing. The invention further provides vectors comprising nucleic acid sequences comprising or consisting of SEQ ID NOs: 1-18, as per the enclosed sequence listing.

The invention further provides a vector encoding the isolated nucleic acids for protein isolation of the invention.

The invention further provides a cell or organism comprising the combination, the nucleic acid molecule(s) or the vector according to the invention as defined above, provided that the cell is not a human germ line cell.

The invention further provides the combination, the nucleic acid molecule(s) or the vector according to the invention as defined above for use in medicine, or for use as a medicament. The invention further provides the combination, the nucleic acid molecule(s) or the vector according to the invention as defined above for use in a method of treating cancer. In one embodiment of the invention, the aforementioned use comprises the use of a chemical inductor of heterodimerization. Preferably, the cells for use in a method of treating cancer are modified T cells. Preferably, the types of cancer to be treated according to the aforementioned use according to the invention include, CML, B-cell lymphoma, acute lymphoblastic leukemia, chronic lymphocytic leukemia, Hodgkins and Non-Hodgkins Lymphomas, mezotelioma, ovarian cancer, prostate cancer etc.

The invention further provides a method for making knockout cells or organisms in more efficient manner where invention provides the combination, the nucleic acid molecule(s) or the vector according to the invention as defined above where genome editing is enhanced by the tethered exonucleases.

The invention further provides a method for making knockin cells or organisms in more efficient manner where invention provides the combination, the nucleic acid molecule(s) or the vector according to the invention as defined above where genome editing is enhanced by the tethered exonucleases and by coaddition of template DNA.

The invention further provides the combination, the nucleic acid molecule(s) or the vector according to the invention as defined above for use as a tool for drug new sources of fuel development etc.

The examples described in more detail below are designed to best describe the invention. These examples do not limit the scope of the invention, but are merely intended to provide a better understanding of the invention and its use.

### Example 1: Preparation of Constructs for Modified CRISPR/Cas System Tethered with Certain Exonucleases Via Different Heterodimerization Systems

Preparation of DNA coding for modified CRISPR/Cas system tethered with certain exonucleases via different heterodimerization systems. In order to prepare DNA constructs, the inventors used molecular biology methods such as: chemical transformation of competent E. coli cells, plasmid DNA isolation, polymerase chain reaction (PCR), reverse transcription—PCR, PCR linking, nucleic acid concentration determination, DNA agarose gel electrophoresis, isolation of fragments of DNA from agarose gels, chemical synthesis of DNA, DNA restriction with restriction enzymes, cutting of plasmid vectors, ligation of DNA fragments, purification of plasmid DNA in large quantities. The exact course of experimental techniques and methods are well known to experts in the field and are described in the manuals of molecular biology.

All the work was performed using sterile techniques, which are also well known to the experts in the field. All plasmids, completed constructs and partial constructs were transformed into the bacteria Escherichia coli by chemical transformation. Plasmids for transfection into cell lines (animal or human) have been isolated using a DNA isolation kit that removes endotoxins.

In the described cases, the Cas9 protein was selected from all types of CRISPR system. Exonucleases were selected among known exonucleases. The Cas9 was connected via a peptide linker with different protein domains of different heterodimerization systems or with peptide from peptide pairs that form coiled-coils at C-terminus. Exonucleases were connected via a peptide linker with different protein domains of different heterodimerization systems or with peptide from peptide pairs that form coiled-coils at N-terminus.

The sequences of Cas9 and certain exonucleases according to the invention are listed in Table 1. All operons were prepared by techniques according to methods known to experts in the field. The operons were inserted into plasmids suitable for eukaryotic systems. The suitability of the nucleotide sequence was confirmed by the inventors by sequencing and restriction analysis.

TABLE 1
Fusion proteins of components of exemplary artificial
engineered NFAT2 transcription factors.

| | | | |
|---|---|---|---|
| 1 | Cas9-P4 | pcDNA3 | Cas9 from Streptococcus pyogenes fused |
| | | | with P4 peptide, that form coiled-coil |
| 2 | Cas9-P3 | pcDNA3 | Cas9 from Streptococcus pyogenes fused |
| | | | with P3 peptide, that form coiled-coil |
| 3 | Cas9-N5 | pcDNA3 | Cas9 from Streptococcus pyogenes fused |
| | | | with N5 peptide, that form coiled-coil |
| 4 | Cas9-N6 | pcDNA3 | Cas9 from Streptococcus pyogenes fused |
| | | | with N6 peptide, that form coiled-coil |
| 5 | Cas9-P3S | pcDNA3 | Cas9 from Streptococcus pyogenes fused |
| | | | with P3S peptide, that form coiled-coil |
| 6 | Cas9-P4S | pcDNA3 | Cas9 from Streptococcus pyogenes fused |
| | | | with P4S peptide, that form coiled-coil |
| 7 | Cas9-AP4 | pcDNA3 | Cas9 from Streptococcus pyogenes fused |
| | | | with AP4 peptide, that form coiled-coil |
| 8 | P4-EXO111 | pcDNA3 | Exonuclease EXOIII, derived from |
| | | | Escherichia coli fused with P4 peptide, |
| | | | that form coiled-coil |
| 9 | P3-EXO111 | pcDNA3 | Exonuclease EXOIII, derived from |
| | | | Escherichia coli fused with P3 peptide, |
| | | | that form coiled-coil |
| 10 | N5-EXO111 | pcDNA3 | Exonuclease EXOIII, derived from |
| | | | Escherichia coli fused with N5 peptide, |
| | | | that form coiled-coil |
| 11 | N6-EXO111 | pcDNA3 | Exonuclease EXO111, derived from |
| | | | Escherichia coli fused with N6 peptide, |
| | | | that form coiled-coil |
| 12 | P3S-EXO111 | pcDNA3 | Exonuclease EXOIII, derived from |
| | | | Escherichia coli fused with P3S peptide, |
| | | | that form coiled-coil |
| 13 | P4S-EXO111 | pcDNA3 | Exonuclease EXOIII, derived from |
| | | | Escherichia coli fused with P4S peptide, |
| | | | that form coiled-coil |
| 14 | AP4-EXO111 | pcDNA3 | Exonuclease EXOIII, derived from |
| | | | Escherichia coli fused with AP4 peptide, |
| | | | that form coiled-coil |
| 15 | P3-TREX2 | pcDNA3 | Exonuclease TREX2, derived from Mus |
| | | | musculus fused with P3 peptide, that |
| | | | form coiled-coil |
| 16 | P4-TREX2 | pcDNA3 | Exonuclease TREX2, derived from Mus |
| | | | musculus fused with P4 peptide, that |
| | | | form coiled-coil |
| 17 | P3-EXO1 | pcDNA3 | Exonuclease EXO1, derived from |
| | | | Homo sapiens fused with P3 |
| | | | peptide, that form coiled-coil |
| 18 | P4-EXO1 | pcDNA3 | Exonuclease EXO1, derived |
| | | | from Homo sapiens fused with P4 |
| | | | peptide, that form coiled-coil |

Methods and techniques of cultivating cell cultures are well known to experts in the field and are therefore briefly described in order to illustrate the implemented examples. Cells from the HEK293T cell line were grown at 37° C. and 5% CO₂. For cultivation, a DMEM medium containing 10% FBS was used, containing all the necessary nutrients and growth factors. Cells K562 were grown in RPMI medium containing 10% FBS at 37° C. and 5% CO₂. When the cell culture reached the appropriate density, the cells were grafted into a new breeding flask and/or diluted. For the use of cells in experiments, the number of cells was determined by a hemocytometer and seeded in density 1×10⁵ per hole into the microtiter plate with 24 holes 18-24 hours before transfection regarding HEK293 cells. The seeded plates were incubated at 37° C. and 5% CO₂ until the cells were 50-70% confluent and ready for transfection by transfection reagent. The transfection was carried out according to the instructions of the transfection reagent manufacturer (e.g., JetPei, Lipofectamine 2000) and was adapted for the microtiter plate used. K562 (1×10⁴) were electroporated with the use of Neon electroporation system in 10 μl electroporation tips. Afterwards the cells were put in RPMI medium, containg 10% FBS in a well of 12 well plate. On the next day the inductor for heterodimerization were added if chemical and non-chemical induced heterodimerization system were used. 48 hours after transfection genomic DNA was isolated and desired area PCR amplified. Next T7E1 (T7 endonuclese 1) assay was performed to determine the percentage of indel mutations.

### Example 2. Defining the Most Successful Exonuclease Cooexpressed with CRISPR/Cas System for Enhacing Genome Editing at eGFP Genomic Region

To detect the most efficient exonuclease cooexpression of certain exonucleases with CRISPR/Cas system was used. HEK293 cell line that stably expresses eGFP was used.

HEK293T, stably expressing eGFP cells, seeded in 24-well plates, were transfected one day prior to the experiment with plasmids, which encode for one of the examined exonuclease and a separate plasmid that encodes CRISPR/Cas system; Cas9 protein from Streptococcus pyogenes and gRNA (targeting sequence in eGFP genomic region: GGCGAGGGCGATGCCACCTA).

To analyze the genome editing activity of cooexpression of certain exonuclease and CRISPR/Cas system, cells were analyzed on flow cytometry 48 hours after transfection. We measured the fluorescence intensity in cell population respectively and presented results in % of MFI (mean fluorescence intensity)

### Results:

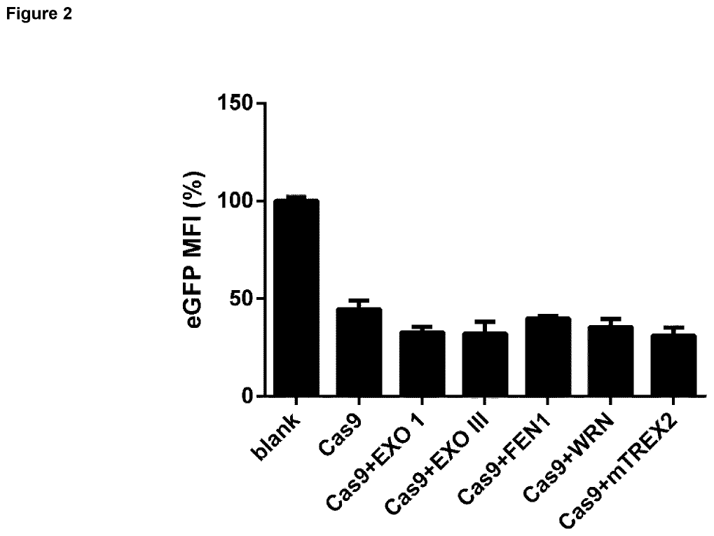

It is clear from FIG. 2 that the cooexpression of certain exonucleases and CRISPR/Cas system did enhance genome editing in eGFP genomic region regarding diminished fluorescence in all cell samples, transfected with CRISPR/Cas system and certain exonuclease. Cooexpression of CRISPR/Cas system targeting eGFP GOI and EXOIII, derived from E.coli showed the most edited percentage of tested cells due to the lowest % of MOI.

### Example 3. Activity of Modified CRISPR/Cas System Tethered with Certain Exonucleases Via Heterodimerizing Peptide Pairs that Form Coiled-Coils

To test the activity of modified CRISPR/Cas system tethered with certain exonucleases via heterodimerizing peptide pairs HEK293 cells were used to determine the activity of modified CRISPR/Cas system tethered with certain exonucleases via heterodimerizing peptide pairs. Cells were transfected with constructs that express CRISPR/Cas system tethered with certain exonucleases via heterodimerizing peptide pairs; for example Cas9-P3 or Cas9-P4 with combination of P3-EXO1 or P4-EXO1 or P3-EXOIII or P4-EXOIII or P3-mTREX2. For control, cells were transfected with regular CRISPR/Cas9 system or were cooexpressed with CRISPR/Cas9 system and certain exonuclease (e.g. EXO1 or EXOIII or mTREX2). gRNA for human EMX1 gene (targeting sequence: GAGTCCGAGCAGAAGAAGAA) or human VEGF gene (targeting sequence: GGTGAGTGAGTGTGTGCGTG) or human MYD88 (GGCTGAGAAGCCTTTACAGG) were used. 48 hours genomic DNA from transfected cells was isolated and genomic region around predicted DSB was PCR amplified. Next T7E1 assay was carried out. Presence and quantification of indel mutation was determined by Syber gold stained PAGE (polyacrylamide gel electrophoresis) gel analysis. Percentage of indel mutations was calculated by ImageJ software. Also next-generation sequencing (NGS) was performed from 150 base pair long amplicons.

### Result:

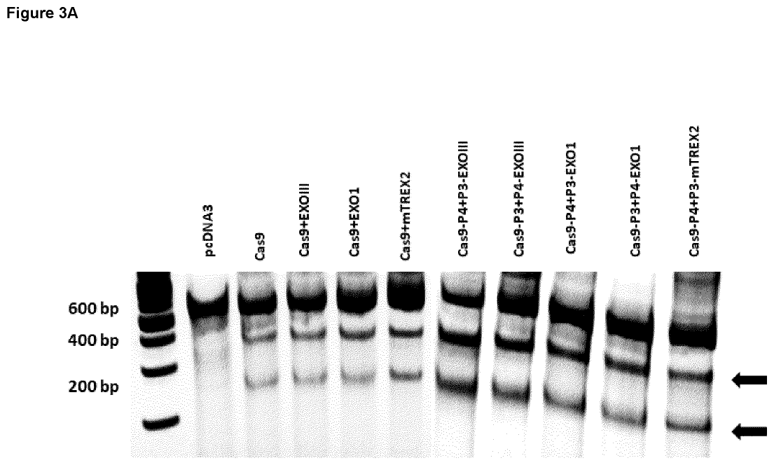

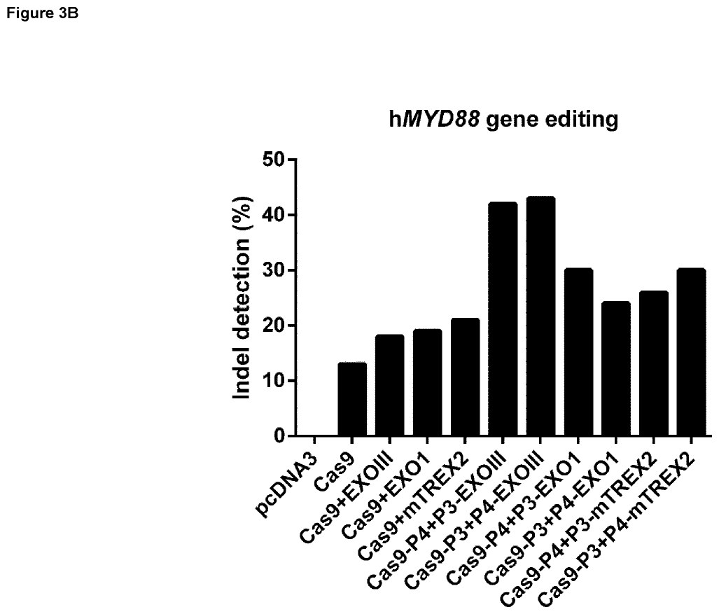

PAGE gel analysis of T7E1 treated samples from transfected cells with appropriate constructs in FIG. 3A indicate that genome editing of human MYD88 gene was highest among all tested samples when Cas9-P4 tethered to EXOIII via P3 peptide was used. A modest decrease in genome editing was observed when cells were transfected with constructs, expressing Cas9-P3 and P4-EXOIII. In all cases, where modified CRISPR/Cas system tethered with certain exonucleases (EXO1 or EXOIII or mTREX2) via heterodimerizing peptide pairs (P3-P4) were used genome editing was higher compared to cell samples with cooexpression of Cas9, gRNA targeting human MYD88 gene and certain exonucleases (e.g. EXO1 or EXOIII or mTREX2). Genome editing, where modified CRISPR/Cas system tethered with certain exonucleases (EXO1 or EXOIII or mTREX2) via heterodimerizing peptide pairs (P3-P4) were used was also higher when regular CRISPR/Cas system was used, where only Cas9 and gRNA targeting human MYD88 gene were used. FIG. 3B graphically shows percentage of indel presence, which were determined with PAGE analysis, depicted in FIG. 3A.

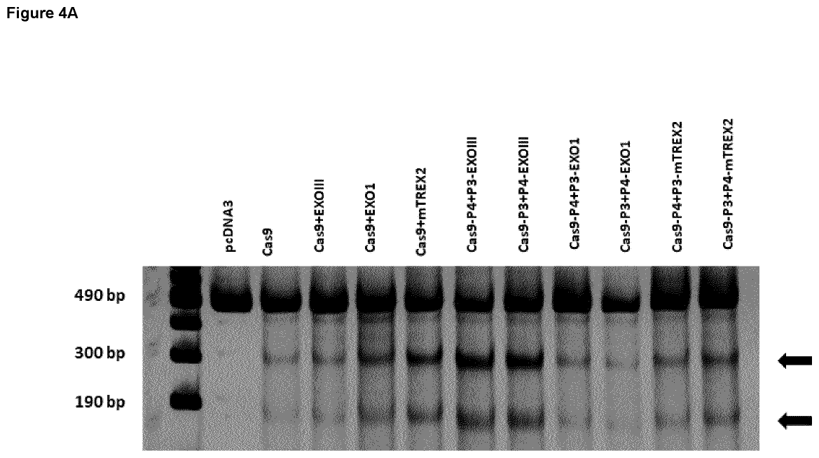

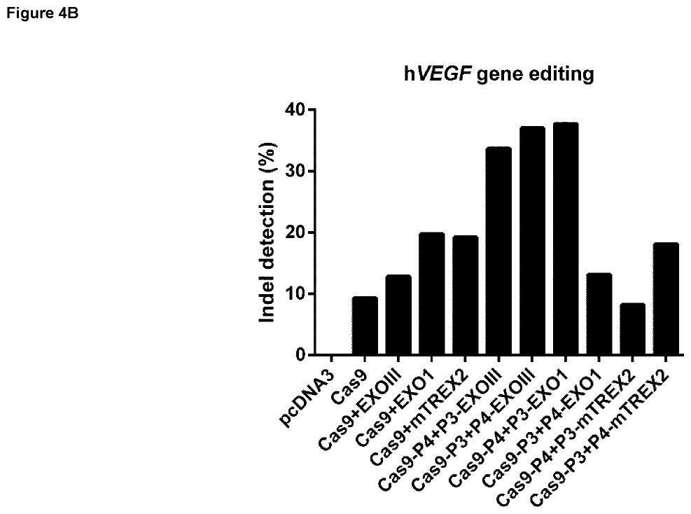

PAGE gel analysis of T7E1 treated samples from transfected cells with appropriate constructs in FIG. 4A indicate that genome editing of human VEGF gene was highest among all tested samples when Cas9-P3 tethered to EXOIII via P4 heterodimerizing peptide was used. A modest decrease in genome editing was observed when cells were transfected with constructs, expressing Cas9-P4 and P3-EXOIII. Genome editing was higher compared to cell samples with cooexpression of Cas9, gRNA targeting human VEGF gene and certain exonucleases (e.g. EXO1 or EXOIII or mTREX2). Genome editing, where modified CRISPR/Cas system tethered with certain exonucleases (EXO1 or EXOIII) via heterodimerizing peptide pairs (P3-P4) were used was also higher when regular CRISPR/Cas system was used, where only Cas9 and gRNA targeting human VEGF gene were used. FIG. 4B graphically shows percentage of indel presence, which were determined with PAGE analysis, depicted in FIG. 4A.

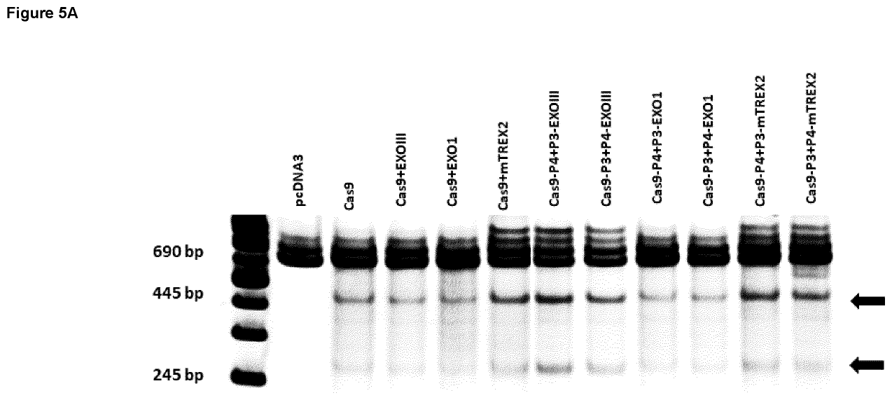

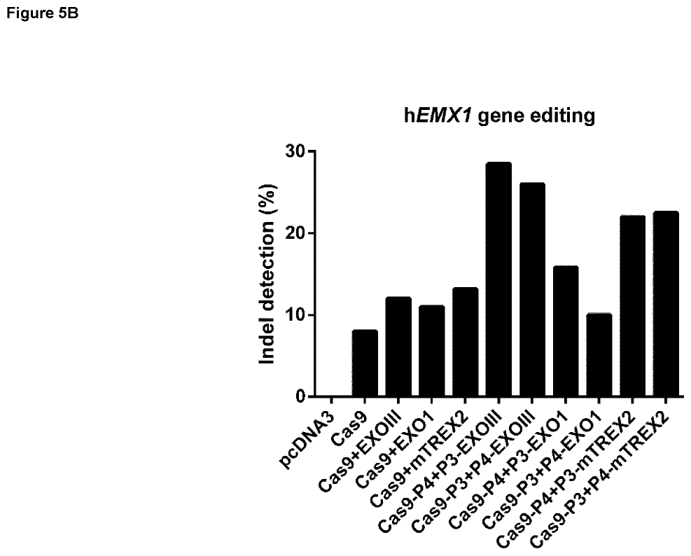

PAGE gel analysis of T7E1 treated samples from transfected cells with appropriate constructs in FIG. 5A indicate that genome editing of human EMX1 gene was highest among all tested samples when Cas9-P4 tethered to EXOIII via P3 peptide was used. A modest decrese in genome editing was observed when cells were transfected with constructs, expressing Cas9-P3 and P4-EXOIII. In all cases, where modified CRISPR/Cas system tethered with certain exonucleases (EXO1 or EXOIII or mTREX2) via heterodimerizing peptide pairs (P3-P4) were used genome editing was higher compared to cell samples with cooexpression of Cas9, gRNA targeting human EMX1 gene and certain exonucleases (e.g. EXO1 or EXOIII or mTREX2). Genome editing, where modified CRISPR/Cas system tethered with certain exonucleases (EXO1 or EXOIII or mTREX2) via heterodimerizing peptide pairs (P3-P4) were used was also higher when regular CRISPR/Cas system was used, where only Cas9 and gRNA targeting human EMX1 gene were used. FIG. 5B graphically shows percentage of indel presence, which were determined with PAGE analysis, depicted in FIG. 5A.

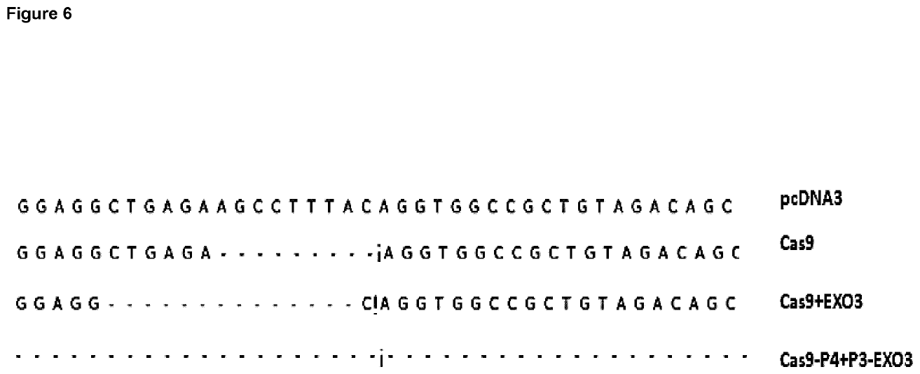

FIG. 6 shows results from NGS. 150 base pair PCR amplicons from cells that were transfected with empty vector pcDNA3 or with regular Cas9 or with Cas9, cooexpressed with EXOIII or with Cas9-P4 and P3-EXOIII were sequenced. In all cases gRNA targeting human MYD88 gene was used. No base pair deletion was observed in sample where only pcDNA3 was expressed. Nine base pair deletion was observed in sample where only Cas9 and gRNA was expressed. Fourteen base pair deletion was observed in sample where Cas9 and gRNA was coexpressed with EXOIII. Forty base pair deletion was observed in sample where Cas9-P4 tethered with P3-EXOIII and gRNA was expressed. Sign (i) depicts the predicted cleavage site for Cas9.

### Example 4: Genome Editing of Modified CRISPR/Cas System Tethered with Certain Exonucleases Via Heterodimerizing Peptide Pairs is not due to the Higher Cas9 Protein Expression

Higher percentage of genome editing for desired GOI can be enhanced due to higher cas9 protein expression. Genome editing of modified CRISPR/Cas system tethered with certain exonucleases via heterodimerizing peptide pairs is not due to the higher Cas9 protein expression.

To experimentally determine that higher genome editing of modified CRISPR/Cas system tethered with certain exonucleases via heterodimerizing peptide pairs is not due to Cas9 protein overexpression we visualized Cas9 protein content via western blot and anti-Cas9 specific immunodetection. To show the same Cas9 protein expression, HEK293 cells were transfected with constructs that express empty pcDNA3 plasmid, CRISPR/Cas system tethered with certain exonucleases via heterodimerizing peptide pairs; for example Cas9-P3 with combination of P4-EXOIII. For control, cells were transfected with regular CRISPR/Cas9 system or were cooexpressed with CRISPR/Cas9 system and certain exonuclese (e.g. EXOIII). gRNA for human MYD88 (target sequence: GGCTGAGAAGCCTTTACAGG) was used. 48 hours cell lysates were prepared. The same amount of protein, determined by standard BCA assay, was analyzed on SDS PAGE gel analysis. Next, western blot transfer was performed and afterwards the membrane was stained with specific anti-Cas9 antibodies.

### Result:

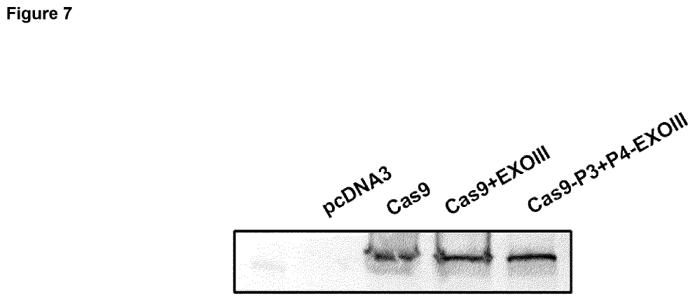

FIG. 7 shows that Cas9 protein content is equal in all tested samples. The band intensity of Cas9 protein is the same for cell lysates of cells, which were transfected with regular CRISPR/Cas9 system, where only Cas9 and gRNA are expressed or for cells that were used for cooexpression of Cas9 and certain exonuclease (e.g. EXOIII) and gRNA and for cells that were transfected with CRISPR/Cas system tethered with certain exonucleases via heterodimerizing peptide pairs; for example Cas9-P3 with combination of P4-EXOIII. In sample where only empty vector was transfected Cas9 protein band is absent.

### Example 5: Higher Genome Editing due to the Modified CRISPR/Cas System Tethered with Certain Exonucleases Via Heterodimerizing Peptide Pairs does not Increase Unwanted Genome Modification at Off-Target Sites

When using CRISPR/Cas system with enhanced functionality there are some possibilities that increased on-target action of CRISPR/Cas bears also additional amplified off-target effect. To show that the modified CRISPR/Cas system tethered with certain exonucleases via heterodimerizing peptide pairs does not increase unwanted genome modification at off-target sites HEK293 cells where transfected with designated constructs for CRISPR/Cas. With standard T7E1 assay 48 hours after transfection potential three off-target sites for MYD88 gRNA (target sequence: GGCTGAGAAGCCTTTACAGG) were analyzed. Due to bioinformatics tool for gRNA design and scoring three potential off-target sites for MYD88 gRNA were predicted, for example: off-target site 1 was found in ANKRD52 gene (target sequence: ACTGTAAAGGCTGCTCTCCC); off-target site 2 was predicted to be in FUT9 gene (target sequence: TGCAGAGGAGCCTTTACATG) and off-target site 3 was determined in PSKH2 gene (target sequence: GCCAGACAAGGCTTTACAGG).

### Result:

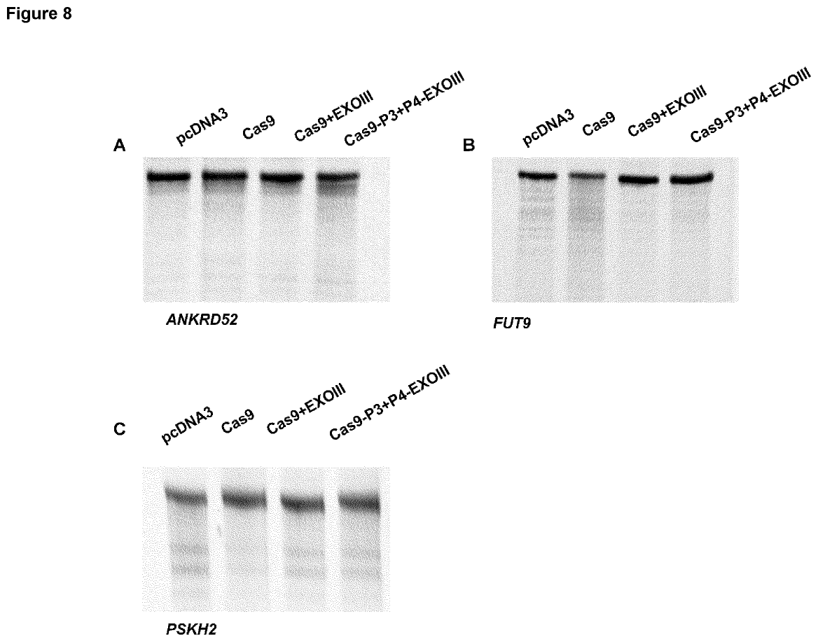

PAGE gel analysis of T7E1 treated samples from transfected cells with appropriate constructs depicted in FIG. 8 indicate that genome editing of three predicted possible off-target sites for human MYD88 gene gRNA (target sequence: GGCTGAGAAGCCTTTACAGG) did not happen. In FIG. 8A PAGE gel analysis of T7E1 treated samples from transfected cells for off-target site 1 that was found in ANKRD52 gene (target sequence: ACTGTAAAGGCTGCTCTCCC) is depicted. There is no indel mutation present due to the absence of DNA bands cleavage. There is no DNA bands cleavage in samples, which were transfected with empty (pcDNA3) vector or with regular CRISPR/Cas9 system, where only Cas9 and gRNA are expressed. No DNA bands cleavage is seen also in cells, that were used for cooexpression of Cas9 and certain exonuclease (e.g. EXOIII) and gRNA and for cells that were transfected with CRISPR/Cas system tethered with certain exonucleases via heterodimerizing peptide pairs; for example Cas9-P3 with combination of P4-EXOIII.

In FIG. 8B PAGE gel analysis of T7E1 treated samples from transfected cells for off-target site 2 that was found in FUT9 gene (target sequence: TGCAGAGGAGCCTTTACATG) is depicted. There is no indel mutation present due to the absence of DNA bands cleavage. There is no DNA bands cleavage in samples, which were transfected with empty (pcDNA3) vector or with regular CRISPR/Cas9 system, where only Cas9 and gRNA are expressed. No DNA bands cleavage is seen also in cells, that were used for cooexpression of Cas9 and certain exonuclease (e.g. EXOIII) and gRNA and for cells that were transfected with CRISPR/Cas system tethered with certain exonucleases via heterodimerizing peptide pairs; for example Cas9-P3 with combination of P4-EXOIII.

In FIG. 8C PAGE gel analysis of T7E1 treated samples from transfected cells for off-target site 3 that was found in PSKH2 gene (target sequence: GCCAGACAAGGCTTTACAGG) is depicted. There are DNA bands cleavage present, but there is also DNA bands cleavage in samples, which were transfected with empty (pcDNA3) vector or with regular CRISPR/Cas9 system, where only Cas9 and gRNA are expressed, thus suggesting presence of SNP in the tested region. DNA bands cleavage are seen also in cells, that were used for cooexpression of Cas9 and certain exonuclease (e.g. EXOIII) and gRNA and for cells that were transfected with CRISPR/Cas system tethered with certain exonucleases via heterodimerizing peptide pairs; for example Cas9-P3 with combination of P4-EXOIII, but the band intensities, which is the criterion for indel mutation determination, are the same in all samples.

### Example 6: Modified CRISPR/Cas System Tethered with Certain Exonucleases Via Heterodimerizing Peptide Pairs Poses Anticancer Therapeutical Properties

Modified CRISPR/Cas system tethered with certain exonucleases via heterodimerizing peptide pairs poses anticancer therapeutical properties when the cause of cancer is in genomic rearrangements. CML (chronic myelogenous leukemia) patients in all cases bear Philadelphia chromosome or BCR-ABL fusion gene (t9, 22; q34;q11) between chromosome 9 and 22. This chromosome has the causative role for coding a hybrid kinase, a tyrosine kinase signaling protein. Constitutive signaling of this kinase is causing cells uncontrollably to divide and proliferate. To determine the possible anti-CML therapeutic option, K562 cells (stably expressing firefly luciferase; K562-fLUC), which carry BCR-ABL fusion gene were electroporated with plasmid DNA of designated CRISPR/Cas9 constructs. gRNA with targeting sequence GACCTGTCTTTTAGACAGGC for leading Cas9 to BCR-ABL fusion gene was used. 72 hours after electroporation of K562-fLUC cells, D-luciferin (Xenogen) was added and bioluminescence was measured by using IVIS® Lumina Series III (Perkin Elmer). Data were analyzed with Living Image® 4.5.2 (Perkin Elmer).

To test the activity of modified CRISPR/Cas system tethered with certain exonucleases via heterodimerizing peptide pairs K562 cells were used to determine the activity of modified CRISPR/Cas system tethered with certain exonucleases via heterodimerizing peptide pairs. Cells were electroporated with constructs that express CRISPR/Cas system tethered with certain exonucleases via heterodimerizing peptide pairs; for example Cas9-P3 or Cas9-P4 or Cas9-N5 with combination of P4-EXOIII or P3-EXOIII or N6-EXOIII. For control, cells were electroporated with regular CRISPR/Cas9 system or were cooexpressed with CRISPR/Cas9 system and certain exonuclease (e.g. EXOIII). gRNA for human BCR-ABL fusion gene (targeting sequence GACCTGTCTTTTAGACAGGC) was used. 48 hours later genomic DNA from cells was isolated and genomic region around predicted DSB was PCR amplified. Next T7E1 assay was carried out. Presence and quantification of indel mutation was determined by Syber gold stained PAGE (polyacrylamide gel electrophoresis) gel analysis. Percentage of indel mutations was calculated by ImageJ software.

### Result:

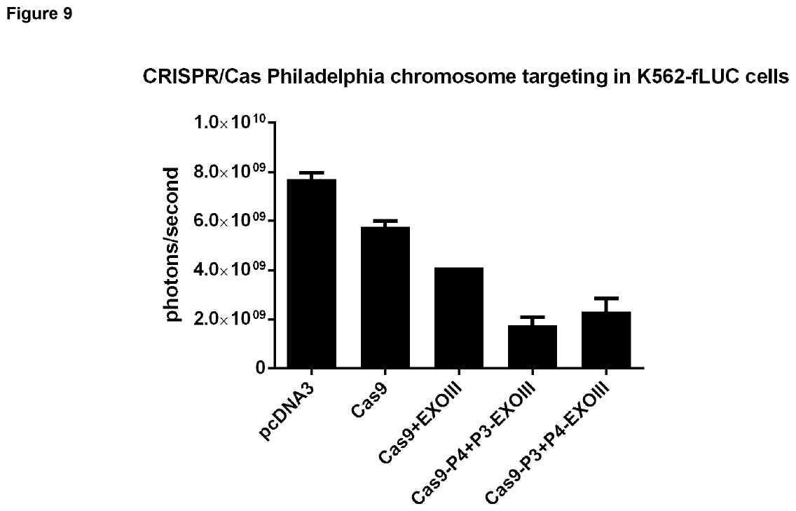

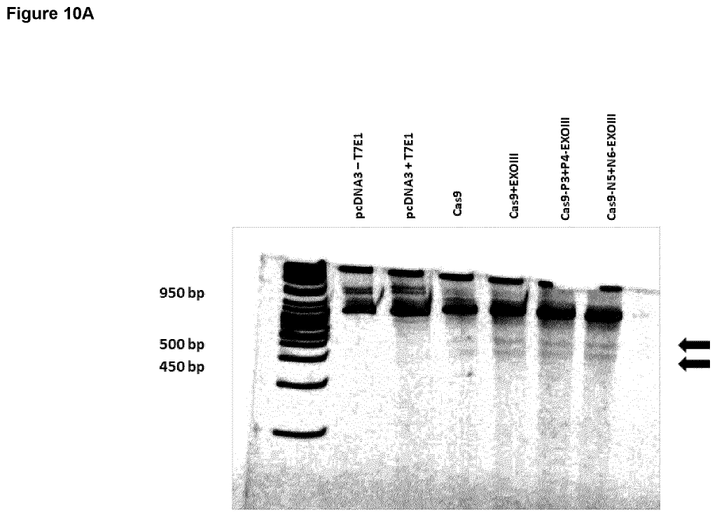

FIG. 9 shows bioluminescence of cell population of K562-fLUC cells after CRISPR/Cas constructs electroporation. Bioluminescence reduction demonstrates cell death due to genomic editing at BCR-ABL fusion gene genomic area. Drop of bioluminescent signal is seen in cells that are electroporated with regular CRISPR/Cas9 system, where only Cas9 and gRNA are expressed, compared to cells, electroporated with empty pcDNA3 vector. Additional drop of bioluminescence is observed when cooexpression of Cas9 and certain exonuclease (e.g. EXOIII) and gRNA is used. The biggest loss of bioluminescent signal due to the highest genome editing at BCR-ABL fusion gene genomic area and consequently cell death is observed in cells that were electroporated with CRISPR/Cas system tethered with certain exonucleases via heterodimerizing peptide pairs; for example Cas9-P3 or Cas9-P4 with combination of P4-EXOIII or P3-EXOIII and corresponding gRNA.

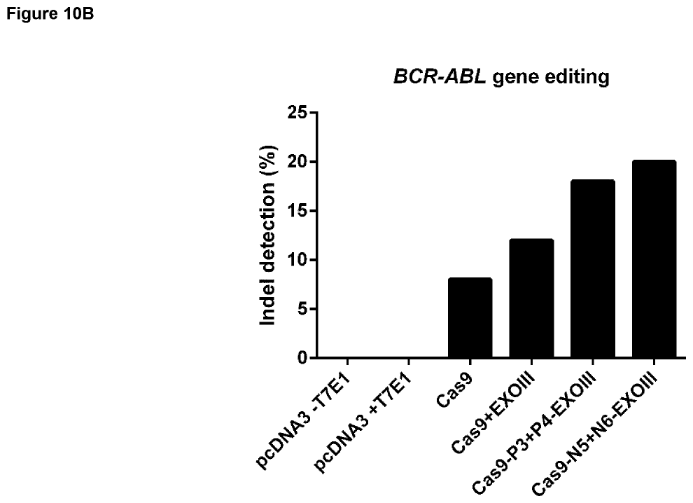

PAGE gel analysis of T7E1 treated samples from transfected cells with appropriate constructs in FIG. 10A indicate that genome editing of human BCR-ABL fusion gene gene was highest among all tested samples when Cas9-N5 tethered to EXOIII via N6 peptide was used. A modest decrease in genome editing was observed when cells were transfected with constructs, expressing Cas9-P3 and P4-EXOIII or in cell samples with cooexpression of Cas9, gRNA targeting human BCR-ABL gene and certain exonucleases (e.g. EXOIII), but nevertheless in all samples genome editing was higher when only Cas9 and gRNA were used. No gene editing occurred when cells were electroporated with empty vector. FIG. 10B graphically shows percentage of indel presence, which were determined with PAGE analysis, depicted in FIG. 10A.

The Invention will be further described by the following items:
1. Modified CRISPR/Cas system tethered with certain exonucleases via heterodimerizing systems comprising two components:
a) a first component comprising a Cas protein, fused with at least one first protein domain of a chemical and non-chemical signal-inducible heterodimerization protein complex, and
b) a second component comprising a certain exonuclease, fused to a second protein domain that binds to the at least one first protein domain of the chemical and non-chemical signal-inducible heterodimerization protein complex.
   2. Modified CRISPR/Cas system tethered with certain exonucleases via heterodimerizing peptide pairs that form coiled-coils comprising two components:
a) a first component comprising a Cas protein, fused with at least one first peptide of peptide pair that form coiled-coils, and
b) a second component comprising a certain exonuclease, fused to a second peptide of peptide pair that form coiled-coils.

3. Modified CRISPR/Cas system tethered with certain exonucleases via heterodimerizing systems of item 1, wherein the at least one first protein domain of the chemical and non-chemical signal-inducible heterodimerization protein complex of the first component is selected from the group consisting of the pairs:
i) FKBP and FRB;
ii) FKBP and calcineurin catalytic subunit A (CnA);
iii) FKBP and cyclophilin;
iv) gyrase B (GyrB) and GyrB;
v) DmrA and DmrC;
vi) ABI and PYL1;
vii) GAI and GID1;
viii) CIB1 and CRY2
ix) LOV and PDZ;
x) PIF and PHYB;
xi) FKF1-GI
xii) UVR8-COP1.
   wherein the second protein domain of the second component is selected from the group consisting of the pairs:
i) FKBP and FRB;
ii) FKBP and calcineurin catalytic subunit A (CnA);
iii) FKBP and cyclophilin;
iv) gyrase B (GyrB) and GyrB;
v) DmrA and DmrC;
vi) ABI and PYL1;
vii) GAI and GID1;
viii) CIB1 and CRY2
ix) LOV and PDZ;
x) PIF and PHYB;
xi) FKF1-GI
xii) UVR8-COP1.
   4. Modified CRISPR/Cas system tethered with certain exonucleases via heterodimerizing peptide pairs of item 2, wherein peptide pairs are selected from any peptides natural and non-natural origin that form coiled-coils.
   5. The Modified CRISPR/Cas system tethered with certain exonucleases via heterodimerizing systems of items 1 and 3 or the modified CRISPR/Cas system tethered with certain exonucleases via heterodimerizing peptide pairs that form coiled-coils of items 2 and 4, uses endonuclease or one or more nickase from any CRISPR type systems.
   6. The Modified CRISPR/Cas system tethered with certain exonucleases via heterodimerizing systems of items 1 and 3 and 5 or the modified CRISPR/Cas system tethered with certain exonucleases via heterodimerizing peptide pairs that form coiled-coils of items 2 and 4-5, uses endonuclease or one or more nickase from any CRISPR type systems that cause one or more DSBs or one or more DNA nicks.
   7. The Modified CRISPR/Cas system tethered with certain exonucleases via heterodimerizing systems of items 1 and 3 and 5-6 or the modified CRISPR/Cas system tethered with certain exonucleases via heterodimerizing peptide pairs that form coiled-coils of items 2 and 4-6, uses any enzyme that recise DNA strands in 3′ and/or 5′ manner.
   8. The Modified CRISPR/Cas system tethered with certain exonucleases via heterodimerizing systems of items 6 and 7 or the modified CRISPR/Cas system tethered with certain exonucleases via heterodimerizing peptide pairs that form coiled-coils of items 6 and 7, influence genome editing at any selected genomic regions.
   9. The Modified CRISPR/Cas system tethered with certain exonucleases via heterodimerizing systems of items 8 or the modified CRISPR/Cas system tethered with certain exonucleases via heterodimerizing peptide pairs that form coiled-coils of items 8, influence genome editing at any selected genomic regions with any designed one or more gRNA.
   10. The modified CRISPR/Cas system tethered with certain exonucleases via heterodimerizing systems of items 8 and 9 or the modified CRISPR/Cas system tethered with certain exonucleases via heterodimerizing peptide pairs that form coiled-coils of items 8 and 9, influence genome editing in prokaryotic and eukaryotic cells.
   11. DSBs developed after function of modified CRISPR/Cas system tethered with certain exonucleases via heterodimerizing systems of items 10 or the modified CRISPR/Cas system tethered with certain exonucleases via heterodimerizing peptide pairs that form coiled-coils of items 10 repair in any cell-repair mechanisms.
   12. The modified CRISPR/Cas system tethered with certain exonucleases via heterodimerizing systems of items 11 or the modified CRISPR/Cas system tethered with certain exonucleases via heterodimerizing peptide pairs that form coiled-coils of items 11 is used for knockin and knockout cell or organisms production.
   13. For knockin production modified CRISPR/Cas system tethered with certain exonucleases via heterodimerizing systems of items 11 or the modified CRISPR/Cas system tethered with certain exonucleases via heterodimerizing peptide pairs that form coiled-coils of items 11 additional DNA template is present.
   14. DNA template of item 13 is present as a ssDNA or dsDNA.
   15. The modified CRISPR/Cas system tethered with certain exonucleases via heterodimerizing systems of items 1 ad 4 and 6-14 comprises a chemical and non-chemical inductor of heterodimerization capable of inducing functional reconstitution of the modified CRISPR/Cas system tethered with exonucleases.
   16. The regulatory system of items 1 and 3, wherein the at least one first protein domain and the second protein domain are from two different proteins and have orthologous properties.
   17. The peptide pairs of item 2 and 4 form coiled-coils in parallel and anti-parallel formation.
   18. The heterodimerizing system of items 1 and 4 and 6-16, wherein the first and second component form a heterodimer in the presence of a chemical inductor of heterodimerization to form the chemical signal-inducible heterodimerization protein complex, wherein the chemical inductor preferably is a small molecule.
   19. The heterodimerizing system of items 1 and 4 and 6-16, wherein the first and second component form a heterodimer in the presence of a non-chemical inductor of heterodimerization to form the non-chemical signal-inducible heterodimerization protein complex, wherein the non-chemical inductor preferably is a light with designated wavelength, appropriate for heterodimerizing system protein partners.
   20. The modified CRISPR/Cas system tethered with certain exonucleases via heterodimerizing systems of items 12 or the modified CRISPR/Cas system tethered with certain exonucleases via heterodimerizing peptide pairs that form coiled-coils of items 12 edits BCR-ABL fusion gene, thereby resulting in decreased deviation and proliferation of cells.
   21. An isolated nucleic acid comprising a nucleic acid encoding a) the first component and/or b) the second component of the with certain exonucleases via heterodimerizing systems of items 1-20 or the modified CRISPR/Cas system tethered with certain exonucleases via heterodimerizing peptide pairs that form coiled-coils of items 1-20.
   22. A vector encoding the isolated nucleic acid(s) of item 21.
   23. Delivery of modified CRISPR/Cas system tethered with certain exonucleases via heterodimerizing systems of items 1-20 or the modified CRISPR/Cas system tethered with certain exonucleases via heterodimerizing peptide pairs that form coiled-coils of items 1-20 as CasRNPs.
   24. A cell or organism comprising the system of items 1-23.
   25. The cell or organism from item 24 for use as a medicament, disese model, drug development model, biofuel production, new food discoveries, method for treating cancer, method for treating genomic mutation derived diseases.
   26. The heterodimerization systems of item 3 and heterodimerizing peptide pairs that form coiled-coils from claim 4 can be used for tethering dCas9 or Cas9 nickase with cytidine deaminase APOBEC.

1-15 (canceled)

16. A combination comprising a first component (a) and a second component
(b), wherein
a) the first component comprises an endonuclease capable of inducing a double stranded DNA break or a nickase capable of inducing a DNA nick, preferably a Cas protein or mutant thereof, fused with a first protein or protein domain, and
b) the second component comprises an exonuclease or a cytidine deaminase, preferably APOBEC protein, fused to a second protein or protein domain, wherein the first and the second components are capable of heterodimerizing with each other via interaction of the first protein or protein domain and the second protein or protein domain, and
wherein the first protein or protein domain and the second protein or protein domain are orthogonal protein partners forming a coiled-coil structure, in particular a coiled-coil structure in parallel or antiparallel orientation.

17. The combination according to claim 16, wherein heterodimerization of the first protein or protein domain and the second protein or domain is inducible via a chemical or non-chemical signal.

18. The combination according to claim 17, wherein the chemical inducing heterodimerization is a small molecule such as rapalog, gibberellin or abscisic acid or any known small molecule.

19. The combination according to claim 17, wherein the non-chemical signal inducing heterodimerization is light with a predetermined wave length.

20. The combination according to claim 16, wherein the first protein or protein domain and the second protein or domain are selected from the group consisting of:
i) FKBP and FRB;
ii) FKBP and calcineurin catalytic subunit A (CnA);
iii) FKBP and cyclophilin;
iv) gyrase B (GyrB) and GyrB;
v) DmrA and DmrC;
vi) ABI and PYL1;
vii) GAI and GID1;
viii) CIB1 and CRY2;
ix) LOV and PDZ;
x) PIF and PHYB;
xi) FKF1-GI; and
xii) UVR8-COP1.

21. The combination according to claim 16, further comprising a donor DNA molecule, which is a single-stranded or double-stranded DNA molecule, particularly a single-stranded DNA molecule, wherein the donor DNA molecule may carry a desired mutation.

22. The combination according to claim 16, wherein the first component comprises Cas9 or Cas9 nickase and the second component comprises an exonuclease, or wherein the first component comprises dCas9 and the second component comprises a cytidine deaminase, preferably APOBEC.

23. A nucleic acid molecule, or a combination of nucleic acid molecules, comprising a first nucleic acid sequence encoding the first component (a) and a second nucleic acid sequence encoding the second component (b) of the combination according to claim 16.

24. A vector comprising the nucleic acid molecule(s) according to claim 23.

25. A cell or non-human organism comprising the combination according to claim 1, nucleic acid molecule(s) comprising a first nucleic acid sequence encoding the first component (a) and a second nucleic acid sequence encoding the second component (b) of the combination according to claim 1, or a vector comprising the nucleic acid molecule(s) comprising a first nucleic acid sequence encoding the first component (a) and a second nucleic acid sequence encoding the second component (b) of the combination according to claim 1, wherein the cell is not a human germ line cell.

26. A medicament comprising
i) a combination as defined in claim 1,
ii) a nucleic acid molecule comprising a first nucleic acid sequence encoding the first component (a) and a second nucleic acid sequence encoding the second component (b) of the combination according to claim 1,
iii) a vector comprising nucleic acid molecule(s) comprising a first nucleic acid sequence encoding the first component (a) and a second nucleic acid sequence encoding the second component (b) of the combination according to claim 1, or
iv) a cell or non-human organism comprising i), ii), or iii).

27. A method for treating a eukaryotic target cell or a eukaryotic target organism, comprising administering the medicament according to claim 26, wherein the eukaryotic target cell is not a human germ line cell and wherein the medicament results in genome editing.

28. The method according to claim 27, wherein the target cell is a vertebrate target cell, in particular a mammalian target cell, preferably a human target cell or wherein the target organism is a mammalian target organism, particularly a human.

29. The method according to claim 28, wherein the vertebrate target cell is an induced or embryonic pluripotent stem cell of a eukaryotic target organism, particularly a human induced or embryonic pluripotent stem cell.

30. The method according to claim 27, wherein the genome editing comprises introducing a donor DNA molecule optionally carrying a desired mutation, into the eukaryotic target cell or eukaryotic target organism, wherein said donor DNA molecule is a single-stranded or double-stranded DNA molecule.

31. The method according to claim 30, wherein said donor DNA molecule is a single-stranded DNA molecule.

32. A method for editing the genome of a eukaryotic target cell or eukaryotic target organism comprising introducing a combination as defined in claim 1, a nucleic acid molecule comprising a first nucleic acid sequence encoding the first component (a) and a second nucleic acid sequence encoding the second component (b) of the combination according to claim 1, or a vector comprising nucleic acid molecule(s) comprising a first nucleic acid sequence encoding the first component (a) and a second nucleic acid sequence encoding the second component (b) of the combination according to claim 1, into a eukaryotic target cell or eukaryotic target organism, wherein the cell is not a human germ line cell.

33. A method for the in vitro editing of a genome of a eukaryotic target cell, comprising introducing a combination as defined in claim 1, a nucleic acid molecule comprising a first nucleic acid sequence encoding the first component (a) and a second nucleic acid sequence encoding the second component (b) of the combination according to claim 1, or a vector comprising nucleic acid molecule(s) comprising a first nucleic acid sequence encoding the first component (a) and a second nucleic acid sequence encoding the second component (b) of the combination according to claim 1, into a eukaryotic target cell, wherein the cell is not a human germ line cell.

34. The method according to claim 33, wherein said eukaryotic target cell is a mammalian target cell.

35. The method according to claim 34, wherein said mammalian target cell is a human target cell.
